(19) United States
(12) Patent Application Publication
(10) Pub. No.: US 2008/0138864 A1
(43) Pub. Date: Jun. 12, 2008

(54) Starch Process

(75) Inventors: Anders Vikso-Nielsen, Slangerup (DK); Sven Pedersen, Gentofte (DK)

Correspondence Address: NOVOZYMES NORTH AMERICA, INC., 500 FIFTH AVENUE, SUITE 1600, NEW YORK, NY, 10110, US

(73) Assignee: NOVOZYMES A/S, BAGSVAERD (DK)

(21) Appl. No.: 11/720,345

(22) PCT Filed: Dec. 12, 2005
(86) PCT No.: PCT/DK05/00783

§ 371 Date: May 29, 2007

(30) Foreign Application Priority Data
Dec. 22, 2004 (DK) PA 2004 01975

(51) Int. Cl. C12P 19/20

(52) U.S. Cl. 435/96

(57) ABSTRACT

The present invention relates, inter alia, to the use of a glucoamylase derived from Talaromyces sp. and an acid alpha-amylase comprising a carbohydrate-binding module in a starch saccharification process in which starch is degraded to glucose.

### FIELD OF THE INVENTION

The present invention relates, inter alia, to the use of a glucoamylase derived from Talaromyces sp. and an acid alpha-amylase comprising a carbohydrate-binding module (“CBM”) in a starch saccharification process comprising degrading starch to glucose.

### BACKGROUND OF THE INVENTION

A thermostable glucoamylase from Talaromyces emersonii is disclosed in WO9928448A1. The purified enzyme shows markedly enhanced stability and a 3-4 fold higher specific activity compared to Aspergillus niger glucoamylase and has optimal activity at pH 4.5 and at 70° C. and thus appears suited for industrial saccharification for production of glucose. The yield of glucose during industrial saccharification with Talaromyces emersonii glucoamylase, however, is 1-2% lower than for Aspergillus niger glucoamylase thereby reducing the enzymes profitability in a process for production of high DX glucose syrups and/or high fructose syrups.

### SUMMARY OF THE INVENTION

Now the inventors of the present invention have surprisingly discovered that in a saccharification process using the Talaromyces glucoamylase a high DX can be reached by the addition of an acid alpha amylase comprising a carbohydrate binding domain (CBM).

Thus the invention provides in a first aspect a process for saccharifying a starch comprising contacting a liquefied starch substrate with a glucoamylase derived from Talaromyces sp. and an acid alpha-amylase comprising a CBM.

In a second aspect the invention provides a process for producing a starch hydrolysate comprising (a) liquefaction, e.g. by jet cooking, with the addition of a thermostable alpha-amylase and (b) subsequently contacting the liquefied starch with an acid alpha-amylase comprising a CBM, and a glucoamylase derived from Talaromyces sp.

The invention provides further embodiments of the two aspects comprising (a) the process wherein the DX (free glucose %) of the hydrolysate following saccharification reaches a value of at least 94.00%, at least 94.50%, at least 94.75% at least 95%, at least 95.25%, at least 95.5%, at least 95.75% or even at least 96%, (b) the process wherein the at least 93%, at least 94%, at least 95%, at least 96%, at least 97%, at least 98%, or preferably at least 99% of the dry solids starch is converted into a soluble hydrolysate, such as e.g. glucose, (c) the process wherein the glucoamylase is a polypeptide having at least 50% homology to the amino acid sequence shown in SEQ ID NO:1, (d) the process wherein the glucoamylase is derived from Talaromyces emersonii, (e) the process wherein the acid alpha-amylase comprising a CBM is a wild type, a variant and/or a hybrid, (f) the process wherein the acid alpha-amylase comprising a CBM is a polypeptide having at least 50% homology to any of the amino acid sequence in the group consisting of SEQ ID NO:2, SEQ ID NO:3, and SEQ ID NO:4, the process wherein the acid alpha-amylase comprising a CBM is present in amounts of 0.05 to 1.0 mg EP/g DS, more preferably from 0.1 to 0.5 mg EP/g DS, even more preferably 0.2 to 0.5 mg EP/g DS of starch, (g) the process wherein the acid alpha-amylase comprising a CBM is present in an amount of 10-10000 AFAU/kg of DS, in an amount of 500-2500 AFAU/kg of DS, or more preferably in an amount of 100-1000 AFAU/kg of DS, such as approximately 500 AFAU/kg DS, (h) the process wherein the glucoamylase is present in amounts of 0.001 to 2.0 AGU/g DS, preferably from 0.01 to 1.5 AGU/g DS, more preferably from 0.05 to 1.0 AGU/g DS, and most preferably from 0.01 to 0.5 AGU/g DS of starch, (i) the process wherein the activities of acid alpha-amylase and glucoamylase are present in a ratio of at least 0.1, at least 0.2, at least 0.25, at least 0.3, at least 0.35, at least 0.40, at least 0.50, at least 0.60, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.85, or even at least 1.9 AFAU/AGU, 0) the process wherein the thermostable alpha-amylase is a bacterial alpha-amylase, preferably derived from a species within Bacillus sp., preferably from a strain of Bacillus licheniformis, (k) the process further comprising adding a debranching enzyme, e.g. a pullulanase or an isoamylase, (l) the process further comprising saccharification to a DX of at least 95 at a temperature from 60° C. to 75° C., preferably from 62° C. to 68° C., more preferably from 64° C. to 66° C., and most preferably 65° C., (m) the process further comprising saccharification to a DX of at least 95 at a temperature from 64° C. to 72° C., preferably from 66° C. to 74° C., more preferably from 68° C. to 72° C., and most preferably 70° C. In a particular embodiment the process further comprises contacting the hydrolysate with a fermenting organism, said fermenting organism preferably a yeast to produce a fermentation product, said fermentation product preferably ethanol, wherein said ethanol is optionally recovered. The saccharification and fermentation may carried out as a simultaneous saccharification and fermentation process (SSF process).

### DETAILED DESCRIPTION OF THE INVENTION

In an embodiment the process of the invention is applied for production of glucose- and/or fructose-containing syrups from starch. The starch may be derived from grain or other starch rich plant parts, preferably corn, wheat, barley, rice, potato. The process may comprise the consecutive enzymatic step; (a) a liquefaction step followed by (b) a saccharification step and optionally (c) (for production of fructose-containing syrups) an isomerization step. During the liquefaction process, starch (initially in the form starch suspension in aqueous medium) is degraded to dextrins (oligo- and polysaccharide fragments of starch), preferably by an thermostable alpha-amylase (EC 3.2.1.1), e.g. a bacterial thermostable alpha-amylase, e.g. a Bacillus licheniformis alpha-amylase (Termamyl™ or Liquozyme X™ available from Novozymes, Denmark), typically at pH values between 5.5 and 6.2 and at temperatures of 95-160″C for a period of approximately 2 hours. After the liquefaction step and before the saccharification step the pH of the medium may be reduced to a value below 4.5 (e.g approximately pH 4.3), maintaining the high temperature (above 95° C.), whereby the liquefying alpha-amylase activity is denatured.

During saccharification the temperature is then normally lowered to below 65° C., such as to 60° C., and the dextrins are converted into dextrose (D-glucose) in the presence of (a) a glucoamylase which according to the invention is derived from Talaromyces and (b) an acid alpha-amylase comprising a CBM. In an embodiment an additional enzyme may be present, preferably a debranching enzyme, such as an isoamylase (EC 3.2.1.68) and/or a pullulanase (EC 3.2.1.41). Preferably the saccharification process allowed to proceed for 24-72 hours until the DX of the hydrolysate reaches a value of at least 94.00%, at least 94.50%, at least 94.75% at least 95%, at least 95.25%, at least 95.5%, at least 95.75% or even at least 96%. Optionally the resulting high DX glucose syrups is converted into high fructose syrup using, e.g., an immobilized “glucose isomerase” (xylose isomerase, EC 5.3.1.5)).

### Alignment and Identity

For purposes of the present invention, alignments of amino acid sequences and calculation of identity scores were done using the software Align, a Needleman-Wunsch alignment (i.e. global alignment), useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Align is from the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98). The relevant part of the amino acid sequence for the identity determination is the mature polypeptide, i.e. without the signal peptide.

### Enzymes

Glucoamylases

Preferred for the invention is any glucoamylase derived from a strain of Talaromyces sp. and in particular derived from Talaromyces leycettanus such as the glucoamylase disclosed in U.S. Pat. No. Re. 32,153, Talaromyces duponti and/or Talaromyces thermopiles such as the glucoamylases disclosed in U.S. Pat. No. 4,587,215 and more preferably derived from Talaromyces emersonii, and most preferably the glucoamylase derived from strain CBS 793.97 and/or disclosed as SEQ ID NO: 7 in WO 99/28448 and as SEQ ID NO:1 herein. Further preferred is a glucoamylase which has an amino acid sequence having at least 50%, at least 60%, at least 70%, at least 80%, at least 90% or even at least 95% identity to the aforementioned amino acid sequence. A commercial Talaromyces glucoamylase preparation is supplied by Novozymes A/S as Spirizyme Fuel.

### Enzymes Having Acid Alpha-Amylase Activity and Comprising a CBM

Preferably the CBM is a starch binding domain (SBD), and preferably the acid alpha-amylase activity is derived from an acid alpha-amylase within EC 3.2.1.1. The enzyme having acid alpha-amylase activity and comprising a CBM to be used in the invention may be a hybrid enzyme or the polypeptide may be a wild type enzyme which already comprises a catalytic module having alpha-amylase activity and a carbohydrate-binding module. The polypeptide to be used in the process of the invention may also be a variant of such a wild type enzyme. The hybrid may be produced by fusion of a first DNA sequence encoding a first amino acid sequences and a second DNA sequence encoding a second amino acid sequences, or the hybrid may be produced as a completely synthetic gene based on knowledge of the amino acid sequences of suitable CBMs, linkers and catalytic domains. The term “hybrid enzyme” is used herein to characterize polypeptides, i.e. enzymes, having acid alpha-amylase activity and comprising a CBM that comprises a first amino acid sequence comprising a catalytic module having alpha-amylase activity and a second amino acid sequence comprising at least one carbohydrate-binding module wherein the first and the second are derived from different sources. The term “source” being understood as e.g. but not limited to a parent polypeptide, e.g. an enzyme, e.g. an amylase or glucoamylase, or other catalytic activity comprising a suitable catalytic module and/or a suitable CBM and/or a suitable linker. The parent polypeptides of the CBM and the acid alpha-amylase activity may be derived from the same strain, and/or the same species or it may be derived from different stains of the same species or from strains of different species. CBM-containing hybrid enzymes, as well as detailed descriptions of the preparation and purification thereof, are known in the art [see, e.g. WO 90/00609, WO 94/24158 and WO 95/16782, as well as Greenwood et al. Biotechnology and Bioengineering 44 (1994) pp. 1295-1305].

Preferred for the invention is any enzyme having acid alpha-amylase activity and comprising a CBM including but not limited to the hybrid enzymes and wild type variants disclosed in PCT/US2004/020499 (NZ10490), and in Danish patent application from Novozymes A/S internal number NZ10729 filed on the same day as the present application. More preferred is an enzyme having acid alpha-amylase activity and comprising a CBM which enzyme has the amino acid sequence disclosed as SEQ ID NO:2 (A.niger+CBM), SEQ ID NO:3 (JA126) or SEQ ID NO:4 (JA129) or any enzyme having acid alpha-amylase activity and comprising a CBM which enzyme which has an amino acid sequence having at least 50%, 60%, 70%, 80%, 90% or even at least 95% identity to any of the aforementioned amino acid sequences.

Preferably the activities of acid alpha-amylase and glucoamylase are present in a ratio of between 0.3 and 5.0 AFAU/AGU. More preferably the ratio between acid alpha-amylase activity and glucoamylase activity is at least 0.35, at least 0.40, at least 0.50, at least 0.60, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.85, or even at least 1.9 AFAU/AGU. However, the ratio between acid alpha-amylase activity and glucoamylase activity should preferably be less than 4.5, less than 4.0, less than 3.5, less than 3.0, less than 2.5, or even less than 2.25 AFAU/AGU.

Methods

### MATERIALS AND METHODS

### Determination of Acid Alpha-Amylase Activity

When used according to the present invention the activity of any acid alpha-amylase may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, i.e., acid stable alpha-amylase, an endo-alpha-amylase (1,4-alpha-D-glucan-glucano-hydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

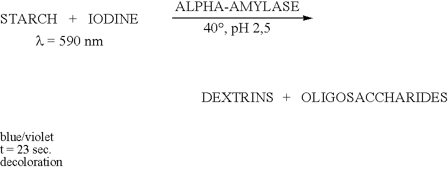

Standard Conditions/Reaction Conditions:

| | |
|---|---|
| Substrate: | Soluble starch, approx. 0.17 g/L |
| Buffer: | Citrate, approx. 0.03 M |
| Iodine (I2): | 0.03 g/L |
| CaCl2: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

A folder EB-SM-0259.02/01 describing this analytical method in more detail is available upon request to Novozymes A/S, Denmark, which folder is hereby included by reference.

### Glucoamylase Activity

Glucoamylase (AMG) activity may be measured in AmyloGlucosidase Units (AGU). The AGU is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG Incubation:

| | |
|---|---|
| Substrate: | maltose 23.2 mM |
| Buffer: | acetate 0.1 M |
| pH: | 4.30 ± 0.05 |
| Incubation | 37° C. ± 1 |
| temperature: | |
| Reaction time: | 5 minutes |
| Enzyme working range: | 0.5-4.0 AGU/mL |

Color Reaction:

| | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### EXAMPLE 1

Substrates for saccharification were prepared by dissolving a DE 11 maltodextrin prepared from corn starch liquefied with thermostable bacterial alpha-amylase (LIQUOZYME X™, Novozymes A/S) in Milli-Q™ water, and adjusting the dry solid matter content (DS) to 30%. The saccharification experiments were carried out in sealed 2 ml glass vials at 60° C. and initial pH of 4.3 under continuous stirring. The following enzymes were used: a Talaromyces emersonii composition (T-AMG), a wild type Aspergillus niger acid alpha-amylase and JA001, which is an alpha-amylase with the same catalytic domain as the wild type A. niger acid alpha-amylase but also comprising a CBM.

Samples were taken at set intervals and heated in boiling water for 15 minutes to inactivate the enzymes. After cooling, the samples were diluted to 5% DS and filtered (Sartorius MINISART™ NML 0.2 μm), before being analysed by HPLC. The glucose levels as a % of total soluble carbohydrate are given in table 1 below.

TABLE 1
The performance of the CBM amylase variant JA001 at two glucoamylase
levels compared with the wild type A. niger acid alpha-amylase, having
the same catalytic module as JA001. Results shown as glucose pct. after
24, 32, 48 and 70 hrs.

| | | | DP1% (glucose) | | | |
|---|---|---|---|---|---|---|
| Enzyme dosage | | | 24 | 32 | | |
| AGU/g DS | AFAU/g DS | | hrs | hrs | 48 hrs | 70 hrs |
| 0.35 | JA001 | 0.0000 | 88.2 | 90.3 | 92.2 | 93.4 |
| | | 0.0875 | 92.0 | 93.6 | 94.9 | 95.5 |
| | | 0.1750 | 93.8 | 94.9 | 95.4 | 95.3 |
| 0.15 | JA001 | 0.0000 | 73.8 | 77.4 | 81.1 | 84.0 |
| | | 0.0875 | 79.2 | 85.8 | 91.4 | 93.9 |
| | | 0.1750 | 88.0 | 92.0 | 94.3 | 95.2 |
| 0.35 | WT A. niger | 0.0875 | 89.8 | 91.9 | 93.5 | 94.4 |
| | Alpha-amylase | 0.1750 | 91.0 | 93.0 | 94.2 | 94.9 |

The results show that the addition of A. niger acid alpha-amylase with Talaromyces emersonii glucoamylase gave a higher glucose yield than with the AMG alone. However the largest effect was seen when the CBM containing acid alpha-amylase variant was added with the T-AMG. The use of the CBM containing acid alpha-amylase variant furthermore allowed reducing the AMG level and still maintaining a high glucose yield.

1-18. (canceled)

19. A process for saccharifying of a starch comprising contacting a liquefied starch substrate with a glucoamylase derived from Talaromyces sp, and an acid alpha-amylase comprising a carbohydrate-binding module.

20. A process for producing a starch hydrolysate, comprising
a) liquefaction with a thermostable alpha-amylase, and
b) subsequently contacting the liquefied starch with
i) an acid alpha-amylase comprising a carbohydrate-binding module, and
ii) a glucoamylase derived from Talaromyces sp.

21. The process of claim 19, wherein the DX of the hydrolysate following saccharifcation is at least 94%.

22. The process of claim 19, wherein at least 93% of the dry solids starch is converted into a soluble hydrolysate.

23. The process of claim 19, wherein the glucoamylase is a polypeptide having at least 50% homology to the amino acid sequence shown in SEQ ID NO: 1.

24. The process of claim 19, wherein the glucoamylase is derived from Talaromyces emersonii.

25. The process of claim 19, wherein the acid alpha-amylase comprising a carbohydrate-binding module is a wild type, a variant and/or a hybrid.

26. The process of claim 19, wherein the acid alpha-amylase comprising a carbohydrate-binding module is a polypeptide having at least 50% homology to any of the amino acid sequence in the group consisting of SEQ ID NO:2, SEQ ID NO:3, and SEQ ID NO:4.

27. The process of claim 19, wherein the acid alpha-amylase comprising a carbohydrate-binding module is present in amounts of 0.05 to 1.0 mg EP/g DS of starch.

28. The process of claim 19, wherein the acid alpha-amylase comprising a carbohydratebinding module is present in an amount of 10-10000 AFAU/kg of DS.

29. The process of claim 19, wherein the glucoamylase is present in an amount of 0.001 to 2.0 AGU/g DS of starch.

30. The process of claim 19, wherein the activities of acid alpha-amylase and glucoamylase are present in a ratio of at least 0.1 AFU/AGU.

31. The process of claim 19, wherein the thermostable alpha-amylase is a bacterial alpha-amylase.

32. The process of claim 19, further comprising adding a debranching enzyme.

33. The process of claim 19, comprising saccharification to a DX of at least 95 at a temperature from 60° C. to 75°C.

34. The process of claim 19, comprising saccharification to a DX of at least 95 at a temperature from 64° C. to 72°C.

35. The process of claim 19, further comprising contacting the hydrolysate with a fermenting organism to produce a fermentation product.

36. The process of claim 19, wherein saccharification and fermentation are carried out as a simultaneous saccharification and fermentation process (SSF process).


SEQUENCE LISTING (4 sequences)

SEQ ID NO: 1 - protein, 591 aa, Talaromyces emersonii
  mat_peptide (1)..(591)
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 2 - protein, 616 aa, Artificial
  A.niger-CBM
AEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 3 - protein, 558 aa, Artificial
  Rhizomucor pusillus amylase with linker and SBD from A. rolfsii
SPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSAGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 4 - protein, 574 aa, Artificial
  Hybrid of Meripilus giganteus amylase with A.rolfsii SBD
RPTVFDAGADAHSLHARAPSGSKDVIIQMFEWNWDSVAAECTNFIGPAGYGFVQVSPPQETIQGAQWWTDYQPVSYTLTGKRGDRSQFANMITTCHAAGVGVIVDTIWNHMAGVDSGTGTAGSSFTHYNYPGIYQNQDFHHCGLEPGDDIVNYDNAVEVQTCELVNLADLATDTEYVRGRLAQYGNDLLSLGADGLRLDASKHIPVGDIANILSRLSRSVYITQEVIFGAGEPITPNQYTGNGDVQEFRYTSALKDAFLSSGISNLQDFENRGWVPGSGANVFVVNHDTERNGASLNNNSPSNTYVTATIFSLAHPYGTPTILSSYDGFTNTDAGAPNNNVGTCSTSGGANGWLCQHRWTAIAGMVGFRNNVGSAALNNWQAPQSQQIAFGRGALGFVAINNADSAWSTTFTTSLPDGSYCDVISGKASGSSCTGSSFTVSGGKLTATVPARSAIAVHTGQKGSGGGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES